(19) United States
(12) Patent Application Publication
(10) Pub. No.: US 2025/0268966 A1
(43) Pub. Date: Aug. 28, 2025

(54) METHOD AND COMPOSITION FOR ENHANCING IMMUNE SYSTEM

(71) Applicant: Zhi Qiao Zhang, Artesia, CA (US)

(72) Inventor: Zhi Qiao Zhang, Artesia, CA (US)

(21) Appl. No.: 18/587,098

(22) Filed: Feb. 26, 2024

(51) Int. Cl. A61K 36/185; A61K 36/282; A61K 36/38; A61K 36/536; A61K 36/539; A61K 36/748; A61P 37/04

(52) U.S. Cl. CPC A61K 36/185; A61K 36/282; A61K 36/38; A61K 36/536; A61K 36/539; A61K 36/748; A61P 37/04

(57) ABSTRACT

A composition for enhancing human immune system may include (i) Herba lysimachiae 0.05-0.3%; (ii) Herba lysimachiae 8-45%; (iii) Herba lysimachiae doederleinii hieron 8-45%; (iv) Folium artmisiae argyi 0.05-0.3%; (v) Herba hedyotis diffusae 0.05-0.3%; (vi) Herba hyperici japonici 5-25%; (vii) Half-branched lotus herba scutelbrice barbata 0.05-0.2%; and (viii) Prunellae vulgaris 10-30%, including water extracted powder or concentrated liquid, paste, etc.

### FILED OF THE INVENTION

This invention relates generally to promoting the healing process of physiological disorders or diseases and, more specifically to nutrient compositions that promote the effectiveness or efficiency of the immune system.

### BACKGROUND OF THE INVENTION

Physiological disorders and diseases affect millions of individuals each year. Severity of such pathological conditions can range from mild discomfort, to severe and permanent debilitating state, to a life-ending terminal disease. Treatments have continually strived to achieve cures that will restore an affected individual to a normal physiological state. For many disorders or diseases, medical treatments are limited to reduction in severity of symptoms or improvement in one's quality of living. Therefore, improvement in the efficacy of an available treatment continues to be an area of medical concern.

The diagnosis and treatment of human diseases similarly continues to be a major area of social concern. As long as there continues to be diseases that affect individuals there will be an effort to understand the cause of such diseases as well as efforts to diagnose and treat such diseases. Preservation of life and reducing the burden on society are two motivating factors that encourage the time and expenditure invested into scientific discovery and development processes. Applying the results of these scientific process to the medical field has led to surprising advancements in medicine which have improved both the quality of life and the life-span of affected individuals.

Even the most beneficial therapeutics available for a particular disorder or disease can result in undesirable side effects. For example, cancer chemotherapies and acquired immune deficiency syndrome (AIDS) treatments cause severe toxic effects due to their lack of specificity or to the occurrence of toxic by-product reactions. Additionally, many times an individual will become resistant to therapeutics during a treatment regime. Numerous other examples of undesirable side effects resulting from therapeutic treatment of, for example, immune-mediated diseases, heart disease, chronic fatigue syndrome, neurodegenerative diseases or an infectious disease also are prevalent and well-known occurrences within the medical field.

The ability to reduce undesirable side effects while maintaining efficacy of a treatment remains an intense area of investigation. Predictability has been hampered in part because of the complexity of the human physiological system and the lack of a complete understanding of physiological mechanisms. For example, the advent of monoclonal antibodies and recombinant DNA technology, each having been hailed as technology that would revolutionize medicine and provide the ability to design highly specific therapeutics for diseases, has yielded less than promising results. Only some 30 years after the discovery of monoclonal antibodies are antibody therapeutics becoming available for therapeutic use. This long delay has resulted from the development of complementary technology that renders the antibodies less toxic to the recipient. Therefore, while beneficial in treating and controlling the impact of physiological disorders and diseases, advances in therapeutic medicines still fall short of providing the sought after cure or the ability to deliver a desired treatment regime to maintain a consistent improvement in quality of living. However, conventional nutritional supplements can only provide a single nutrient, so it is not very efficient for the immune system to clear pathogens.

### SUMMARY OF THE INVENTION

It is a main object of the present invention to provide a method and nutritional composition to enhance the immune system.

The present invention aims to overcome the constraints, duration issues, lack of universality, and potential side effects associated with current technologies affecting the immune system. It specifically targets the monitoring, defense, and regulation aspects of the immune system's functions.

It is another object of the present invention to provide nutrient compositions that can not only enhance human immune system, but regulate cholesterol levels.

In one aspect, a composition for enhancing human immune system may include (i) Herba lysimachiae 0.05-0.3%; (ii) Herba lysimachiae 8-45%; (iii) Herba lysimachiae doederleinii hieron 8-45%; (iv) Folium artmisiae argyi 0.05-0.3%; (v) Herba hedyotis diffusae 0.05-0.3%; (vi) Herba hyperici japonici 5-25%; (vii) Half-branched lotus herba scutelbrice barbata 0.05-0.2%; and (viii) Prunellae vulgaris 10-30%, including water extracted powder or concentrated liquid, paste, etc.

In one embodiment, the composition for enhancing human immune system may include (i) Herba lysimachiae 0.03-0.25%; (ii) Herba lysimachiae 10-35%; (iii) Herba lysimachiae doederleinii hieron 10-35%; (iv) Folium artmisiae argyi 0.03-0.25%; (v) Herba hedyotis diffusae 0.03-0.25%; (vi) Herba hyperici japonici 7-28%; (vii) Half-branched lotus herba scutelbrice barbata 0.03-0.25%; and (viii) Prunellae vulgaris 5-25%, including water extracted powder or concentrated liquid, paste, etc.

In another embodiment, the composition for enhancing human immune system may include (i) Herba lysimachiae 0.07-0.25%; (ii) Herba lysimachiae 6-33%; (iii) Herba lysimachiae doederleinii hieron 6-33%; (iv) Folium artmisiae argyi 0.07-0.25%; (v) Herba hedyotis diffusae 0.07-0.25%; (vi) Herba hyperici japonici 8-30%; (vii) Half-branched lotus herba scutelbrice barbata 0.03-0.25%; and (viii) Prunellae vulgaris 8-27%, including water extracted powder or concentrated liquid, paste, etc.

Further technical features and advantages of the present invention will be described in the following description of exemplary embodiments and the appended claims of the present invention.

### BRIEF DESCRIPTION OF THE DRAWINGS

The present invention will be illustrated in more detail below on the basis of exemplary embodiments shown in the accompanying drawings, wherein:

FIG. 1 illustrates a first tester's medical record before taking the nutrient compositions in the present invention.

FIG. 2 illustrates the first tester's medical record after taking the nutrient compositions in the present invention.

FIG. 3 illustrates a second tester's medical record before taking the nutrient compositions in the present invention.

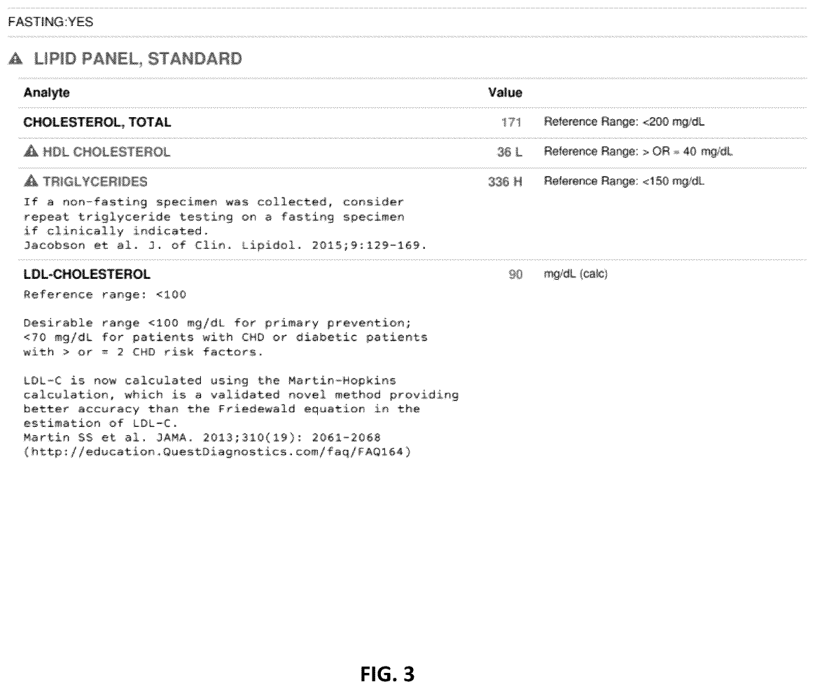

FIG. 4 illustrates the second tester's medical record after taking the nutrient compositions in the present invention.

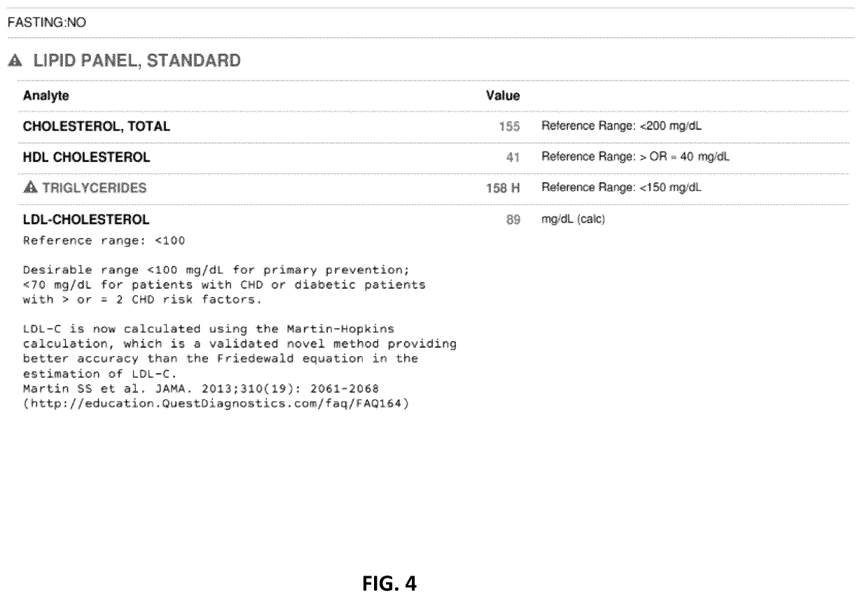

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

This invention is directed to nutrient formulations that promote the effectiveness or efficiency of an immune system. The nutrient formulations also promote the effectiveness or efficiency of mitochondrial functions leading to enhanced energy production, detoxification functions of detoxifying organs and decreasing free radical generation or other oxidative processes. The nutrient formulations have the advantage in that they can be used in combination with other therapies to augment concurrent treatment or they can be used alone on healthy or diseased individuals to promote the health state or delay onset or severity of a disease.

In one aspect, a composition for enhancing human immune system may include (i) Herba lysimachiae 0.05-0.3%; (ii) Herba lysimachiae 8-45%; (iii) Herba lysimachiae doederleinii hieron 8-45%; (iv) Folium artmisiae argyi 0.05-0.3%; (v) Herba hedyotis diffusae 0.05-0.3%; (vi) Herba hyperici japonici 5-25%; (vii) Half-branched lotus herba scutelbrice barbata 0.05-0.2%; and (viii) Prunellae vulgaris 10-30%, including water extracted powder or concentrated liquid, paste, etc.

In one embodiment, the composition for enhancing human immune system may include (i) Herba lysimachiae 0.03-0.25%; (ii) Herba lysimachiae 10-35%; (iii) Herba lysimachiae doederleinii hieron 10-35%; (iv) Folium artmisiae argyi 0.03-0.25%; (v) Herba hedyotis diffusae 0.03-0.25%; (vi) Herba hyperici japonici 7-28%; (vii) Half-branched lotus herba scutelbrice barbata 0.03-0.25%; and (viii) Prunellae vulgaris 5-25%, including water extracted powder or concentrated liquid, paste, etc.

In another embodiment, the composition for enhancing human immune system may include (i) Herba lysimachiae 0.07-0.25%; (ii) Herba lysimachiae 6-33%; (iii) Herba lysimachiae doederleinii hieron 6-33%; (iv) Folium artmisiae argyi 0.07-0.25%; (v) Herba hedyotis diffusae 0.07-0.25%; (vi) Herba hyperici japonici 8-30%; (vii) Half-branched lotus herba scutelbrice barbata 0.03-0.25%; and (viii) Prunellae vulgaris 8-27%, including water extracted powder or concentrated liquid, paste, etc.

Following the implementation of this technology, a comparative analysis of immune cells demonstrates an increase in both numerical values and functional capabilities. Specifically, immune T cells, immunoglobulins, platelets, B cells (producing antibodies), white blood cells, and neutrophils exhibit heightened numbers and enhanced functionalities. The derived benefits encompass a decrease in HBV virus levels, restoration of liver function, reduction in liver tumor size, elimination of HIV symptoms, lowered uric acid and cholesterol levels, regulation of endocrine disorders, mitigation or elimination of associated symptoms, all achieved without any observed side effects.

### Example 1

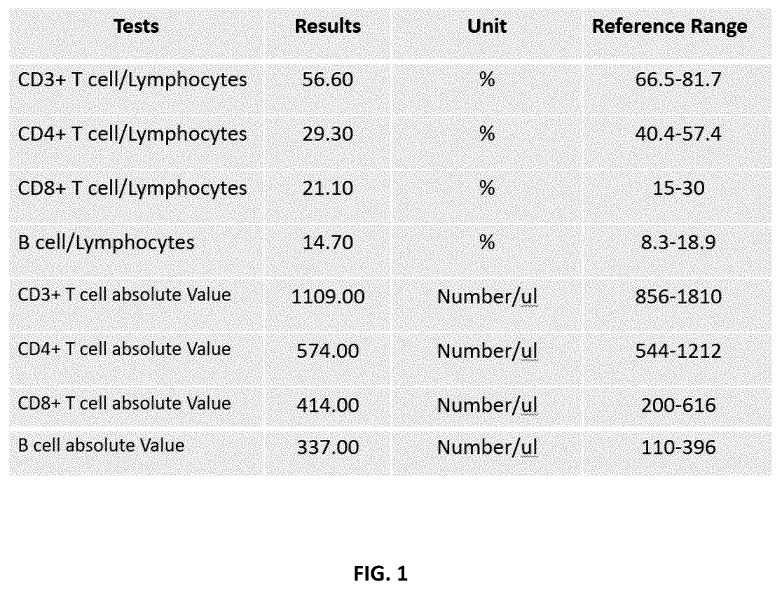

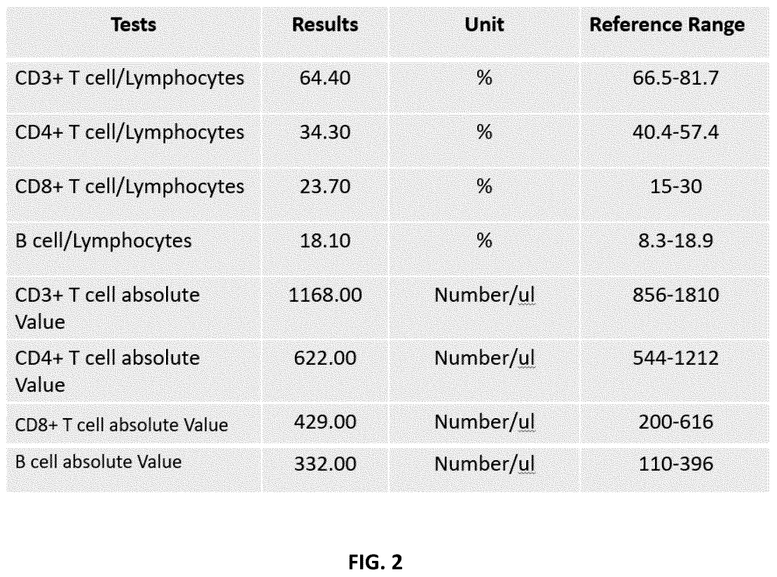

For the first tester, the absolute values of B cell, CD3+ T cell, CD4+ T cell and CD8+ T cell are 14.7, 1109, 574, and 414, respectively before taking the nutrient compositions in the present invention as shown in FIG. 1. The first tester then took the nutrient compositions in the present invention for two months, and the absolute values of CD3+ T cell, CD4+ T cell and CD8+ T cell increase to 18.1, 1168, 622 and 429 respectively as shown in FIG. 2. Thus, it is believed that the nutrient compositions in the present invention can enhance human immune system.

### Example 2

The nutrient compositions in the present invention can not only be used for enhancing human immune system, but also for regulating cholesterol levels. For the second tester, the HDL cholesterol is 36 (Reference Range: > or =40) and Triglycerides is 336 (Reference Range: < or =150) before taking the nutrient compositions in the present invention. After taking the nutrient compositions in the present invention, the number becomes HDL cholesterol is 41 and Triglycerides is 158. Even though the Triglycerides is still higher than the reference range, but it has decreased more than 50%. Thus, it is believed that the nutrient compositions in the present invention can not only enhance human immune system, but also regulate cholesterol levels.

### Example 3

The nutrient compositions in the present invention can also be used for curing HBV viral hepatitis. The tester was infected with HBV viral hepatitis, and before administrating the nutrient compositions in the present invention, the HBV-DNA value was 9.6E+06 (meaning 9.6×106), and the testing continued to administrate the nutrient compositions in the present invention for six months, the HBV-DNA value was reduced to 1.41E+04. Namely, the HBV-DNA value was reduced more than 500 times comparing from the original number.)

The nutrient compositions in the present invention can not only be used for curing HBV viral hepatitis, but regulating the liver function. The ALT and AST values were 76, 53 respectively when the tester was infected with HBV viral hepatitis, however, after being administrated with the nutrient compositions in the present invention for six months, the values were reduced to 20 and 23, respectively.

### Example 4

Similar results can be found on another tester who was also infected with HBV viral hepatitis. Before administrating the nutrient compositions in the present invention, the HBV-DNA value was 5.323E+05, and after being administrated with the nutrient compositions in the present invention for about two years, the HBV-DNA value went down to 7.49E+03, while the ALT and AST values were 38, 24 respectively in the beginning, and went down to 26, 22 respectively after about two years. Thus, it is believed that nutrient compositions in the present invention can not only be used to cure HBV viral hepatitis, but regulate liver function.

Preferred embodiments of the present invention also contain other suitable components in addition to the aforesaid components. Although preferred embodiments of the present invention have been disclosed herein, it should be understood that the foregoing embodiments and technical features are provided as examples only and are not intended to limit the present invention. Various changes and substitutions can be made by those skilled in the art without departing from the invention. Accordingly, the present invention should be construed broadly to cover all such changes, modifications and alternative embodiments within the spirit and scope of the appended claims hereinafter.

What is claimed is:

1. A composition for enhancing human immune system comprising:
(i) Herba lysimachiae 0.05-0.3%;
(ii) Herba lysimachiae 8-45%;
(iii) Herba lysimachiae doederleinii hieron 8-45%;
(iv) Folium artmisiae argyi 0.05-0.3%;
(iv) Herba hedyotis diffusae 0.05-0.3%;
(v) Herba hyperici japonici 5-25%;
(vi) Half-branched lotus herba scutelbrice barbata 0.05-0.2%; and
(vii) Prunellae vulgaris 10-30%, including water extracted powder or concentrated liquid, paste, etc.

2. The composition for enhancing human immune system of claim 1, wherein the weight percentage of Herba lysimachiae can be 10-35%.

3. The composition for enhancing human immune system of claim 1, wherein the weight percentage of Herba lysimachiae doederleinii hieron can be 10-35%.
